(19) United States
(12) Patent Application Publication
(10) Pub. No.: US 2023/0165919 A1
(43) Pub. Date: Jun. 1, 2023

(54) ANTI-OBESITY FUNCTIONAL FEED COMPOSITION CONTAINING HIBISCUS MANIHOT L. EXTRACT AS ACTIVE INGREDIENT FOR COMPANION ANIMALS

(71) Applicant: Andong National University Industry-Academic Cooperation Foundation, Andong-si (KR)

(72) Inventor: Jin-Boo JEONG, Andong-si (KR)

(73) Assignee: Andong National University Industry-Academic Cooperation Foundation, Andong-si (KR)

(21) Appl. No.: 17/578,505

(22) Filed: Jan. 19, 2022

(30) Foreign Application Priority Data
Nov. 29, 2021 (KR) 10-2021-0166732

(51) Int. Cl. A61K 36/185; A61P 3/04

(52) U.S. Cl. CPC A61K 36/185; A61P 3/04; A61K 2236/333; A61K 2236/37; A61K 2236/331

(57) ABSTRACT

Proposed is an anti-obesity functional feed composition for companion animals, the composition including Geumhwagyu (Hibiscus manihot L.) extract as an active ingredient. The Geumhwagyu (Hibiscus manihot L.) extract induces the degradation of CEBPα (CCAAT/enhancer-binding protein-α), thereby inhibiting the differentiation of preadipocytes. In addition, the Geumhwagyu (Hibiscus manihot L.) extract inhibits the expression of peroxisome proliferator-activated receptor-γ (PPARγ), CCAAT/enhancer-binding protein-α (CEBPα), perilipin-1, adiponectin, fatty acid binding protein-4 (FABP4), etc., thereby inhibiting the accumulation of lipids in adipocytes. Thus, the Geumhwagyu (Hibiscus manihot L.) extract shows anti-obesity activity by lowering the content of triacylglycerol (TG).

### CROSS REFERENCE TO RELATED APPLICATION

The present application claims priority to Korean Patent Application No. 10-2021-0166732, filed Nov. 29, 2021, the entire contents of which is incorporated herein for all purposes by this reference.

### BACKGROUND OF THE INVENTION

### 1. Field of the Invention

The present disclosure relates to an anti-obesity functional feed composition for companion animals, the composition including Hibiscus manihot L. extract as an active ingredient.

### 2. Description of the Related Art

Obesity refers to a medical condition in which body fat is excessively accumulated to the extent that it may have a negative effect on health. When the number of calories taken from food exceeds the number of calories consumed through physical activity, it is stored as body fat. Obesity for men is a condition in which the body fat accounts for 25% or more of the weight, and obesity for women is a condition in which the body fat accounts for 30% of the weight. Obesity is a state in which fat is excessively accumulated in the body and is a risk factor that causes various adult diseases as well as appearance problems. Obesity is a cause of fatty liver, hyperlipidemia, osteoarthritis, cholelithiasis, hypertension, diabetes, and cardiovascular disease. In addition, it is known that prostate cancer, rectal cancer, and colorectal cancer are associated with obesity in men, and breast cancer, ovarian cancer, and uterine cancer are associated with obesity in women. In particular, obesity is a significant risk factor for heart disease, and it has been reported that obesity can significantly affect the structure and function of the heart. As it is known that the risk of heart failure increases rapidly with obesity in childhood, it is becoming an important problem in health care to solve obesity. The causes of obesity include diseases such as abnormalities in hypothalamic function and energy metabolism, and genetic factors, but most obesity is caused by lifestyle habits caused by excessive nutritional intake and a decrease in the amount of physical activity. In recent years, the incidence of obesity has continued to increase due to the increase in consumption of instant food common in modern dietary patterns, lack of exercise, and more convenient lifestyle. In addition, it is predicted that this trend will become more severe as time goes by. Recently, obesity has been socially recognized as a disease, and various pharmaceutical companies are focusing their efforts on developing drugs that treat obesity. One of the fat absorption inhibitors, “Xenical™” (Roche Pharmaceuticals, Switzerland), is one of the most used obesity treatments worldwide. Orlistat, a component of Xenical, binds with digested fat and inhibits absorption of the fat in the intestine, thereby causing a portion of the fat that is taken through a meal to be excreted without being absorbed in the body. Aside from this, there are satiety-enhancing drugs including Reductil™ (Abbott, USA), Exolise™ (Atopharma, France), etc. However, it has been reported that the commercialized drugs exhibit side effects including liver damage, gastrointestinal bleeding, gastroenteritis, and kidney stones. In addition, the drugs have a risk of causing heart disease, respiratory disease, and nervous system disease. Therefore, there is a need to develop anti-obesity drugs having excellent anti-obesity effects while exhibiting fewer side effects attributable to problems such as stability as described above.

Geumhwagyu (Hibiscus manihot L.) is a precious medicinal plant all parts of which, such as roots, stems, leaves and flowers, exhibit medicinal properties. It can be obtained during the harvest period ranging from July to August. Since Hibiscus manihot L. is rich in collagen, it is effective for skin care. In addition, since Hibiscus manihot L. contains a large amount of useful ingredients such as palmitic acid, gocifetin, oleic acid, betaine, and linolenic acid, it is also popularly used as a flower tea.

The present inventors of the present patent application have researched and studied on physiological activities of the Geumhwagyu (Hibiscus manihot L.) and has found that the Geumhwagyu (Hibiscus manihot L.) extract has an anti-obesity effect.

### DOCUMENT OF RELATED ART

(Patent Document 0001) Korean Patent Application Publication No. 10-2020-0120465(Title: Composition for Hangover Relief and for Preventing or Treating Alcohol-induced Liver Damage, Applicant: Solgo Biomedical Co., Ltd., Date of Publication: Oct. 21, 2020)
(Patent Document 0002) Korean Patent No. 10-2176935 (Title: Cosmetic Composition for Wrinkle Improvement and Anti-inflammatory, Applicant: Jong-bok Kwak, Date of Registration: Nov. 4, 2020)

### SUMMARY OF THE INVENTION

An objective of the present disclosure is to provide an anti-obesity functional feed composition for companion animals, the composition including Geumhwagyu (Hibiscus manihot L.) extract as an active ingredient.

The present disclosure relates to an anti-obesity functional feed composition for companion animals, the composition including Geumhwagyu (Hibiscus manihot L.) extract as an active ingredient.

The Hibiscus manihot L. extract may be extract extracted from flowers, stems, leaves, or roots of Geumhwagyu (Hibiscus manihot L.) plants. In addition, the Geumhwagyu (Hibiscus manihot L.) extract may be obtained through an extraction process using water, C₁₋₄ alcohol, or a mixed solution thereof as a solvent. The Geumhwagyu (Hibiscus manihot L.) extract may be filtrate obtained by adding Geumhwagyu (Hibiscus manihot L.) to water, C₁₋₄ alcohol, or a mixed solution thereof, stirring or dipping (i.e., extracting) at 4° C. to 50° C. for 1 to 5 days, and filtering the resulting product. Alternatively, the Geumhwagyu (Hibiscus manihot L.) extract may be a dried product obtained by drying the filtrate. More preferably, the solvent may be water.

The Geumhwagyu (Hibiscus manihot L.) extract is preferably treated to have at an effective concentration of 10 to 200 μg/ml so as to be used as an anti-obesity composition. Preferably The Geumhwagyu (Hibiscus manihot L.) extract may have a concentration of 10 to 100 μg/ml and more preferably a concentration of 50 to 100 μg/ml. The C₁₋₄ alcohol may be any one selected from the group consisting of methanol, ethanol, propanol, isopropanol, butanol, and isobutanol.

The filtrate may be dried and powdered. The filtrate may be dried through conventional drying methods such as freeze drying, hot air drying, spray drying, and the like to become powder.

In addition, as a conventional method used in the related art, after the extract that is extracted using water, C₁₋₄ alcohol, or a mixed solution of water and C₁₋₄ alcohol, the extract may be dissolved in water and then fractionated using one or more solvents selected from the group consisting of n-hexane, methylene chloride, acetone, chloroform, ethyl acetate, and n-butanol so that the extract may be provided as fractions.

Alternatively, Geumhwagyu (Hibiscus manihot L.) may be extracted with water, C₁₋₄ alcohol, or a mixed solution of water and C₁₋₄ alcohol to prepare the Geumhwagyu (Hibiscus manihot L.) extract, and water may be added to the extract so that the extract becomes a suspension. Here, the water may be added in an amount of 1 to 1000 times the weight of the extract, and more preferably the water may be added in an amount of 1 to 50 times the weight of the extract. Next, a solvent selected from the group consisting of hexane, chloroform, ethyl acetate, and butanol may be added to the suspension to produce a Geumhwagyu (Hibiscus manihot L.) fraction. The Geumhwagyu (Hibiscus manihot L.) fraction is preferably prepared as concentrate of a hexane layer, concentrate of a chloroform layer, concentrate of an ethyl acetate layer, concentrate of a butanol layer, or concentrate of water. The concentrate of a hexane layer is prepared by: suspending Geumhwagyu (Hibiscus manihot L.) extract obtained by extracting Geumhwagyu (Hibiscus manihot L.) with water, C₁₋₄ alcohol, or a mixed solution thereof and concentrating the resulting extract; suspending the extract on water, and mixing hexane with the extract. The concentrate of a chloroform layer is prepared by removing the hexane layer and mixing chloroform with the residue (i.e., water layer). The concentrate of an ethyl acetate layer is prepared by removing the chloroform layer and mixing ethyl acetate with the residue (i.e., water layer). The concentrate of a butanol layer is obtained by removing the ethyl acetate layer and mixing butanol with the residue (i.e., water layer). The concentrate of a water layer is obtained by removing the butanol layer. On the other hand, other fractionation conditions are not limited except for such a fraction preparation method in which a suspension is prepared by adding water in an amount of 1 to 50 times the weight of the Geumhwagyu (Hibiscus manihot L.) extract to the Geumhwagyu (Hibiscus manihot L.) extract, and adding a solvent selected from the group consisting of hexane, chloroform, ethyl acetate, and butanol, in the same amount as the water. In addition, when adding chloroform to the residue after removing the hexane layer, when adding ethyl acetate to the residue after removing the chloroform layer, and when adding butanol to the residue after removing the ethyl acetate layer, the solvent that is chloroform, ethyl acetate, or butanol may be added in the same amount as the residue to prepare the fraction whenever the solvent is sequentially added.

As the solvent used for extracting the Geumhwagyu (Hibiscus manihot L.) to obtain the Geumhwagyu (Hibiscus manihot L.) extract, it is more preferable to use water rather than an organic solvent.

As an apparatus for preparing the Geumhwagyu extract or fractions, a widely used common extraction apparatus, an ultrasonic crushing extractor, or a fractionator may be used. The prepared Geumhwagyu (Hibiscus manihot L.) extract may be dried with hot air, dried under reduced pressure, or freeze-dried to remove the solvent. In addition, the Geumhwagyu (Hibiscus manihot L.) extract or fraction may be purified through column chromatography.

The Geumhwagyu (Hibiscus manihot L.) extract may be used after fractionation or purification using one or more known methods that are used for separation and extraction of plant components, including extraction with an organic solvent (alcohol, ether, acetone, etc.), distribution of hexane and water, and column chromatography.

The chromatography may be selected from the group consisting of silica gel column chromatography, LH-20 column chromatography, ion exchange resin chromatography, medium pressure liquid chromatography, thin layer chromatography, silica gel vacuum liquid chromatography, and high performance liquid chromatography.

The feed composition of the present invention may include various excipients included in a conventional feed composition for companion animals. Examples of the excipient include minerals such as carbohydrates, proteins, fats, vitamins, and minerals. Sources of carbohydrates, proteins, fats, etc. include various grains, meat, fish, and seafood. As the grains, rice (brown or polished rice), barley, wheat, etc. may be used. As the meet, chicken, duck, beef, pork, sheep, venison, and/or horse meat may be used. As the fish, anchovies, frozen pollack, mackerel, mackerel pike, salmon, tuna, and bonito may be used. As the sea food, abalone, scallops, mussels, etc. may be used. All of these are largely independent of the type. The minerals may include macrominerals, trace minerals, etc. The macrominerals may include calcium, phosphorus, sodium, potassium, chlorine, sulfur, magnesium, etc. The trace minerals may include iron, zinc, iodine, copper, selenium, manganese, cobalt, chromium, fluorine, molybdenum, etc. Other trace minerals may include silicon, vanadium, nickel, tin, cadmium, arsenic, aluminum, boron, and the like. All of these minerals and minerals that can be used may not be particularly limited by their type.

The companion animal may be a dog, cat, bird, rabbit, parrot, hedgehog, hamster, squirrel, or guinea pig.

The present disclosure relates to an anti-obesity functional feed composition for companion animals, the composition including Geumhwagyu (Hibiscus manihot L.) extract as an active ingredient. The Geumhwagyu (Hibiscus manihot L.) extract induces the degradation of CEBPα (CCAAT/enhancer-binding protein-α), thereby inhibiting the differentiation of preadipocytes. In addition, the Geumhwagyu (Hibiscus manihot L.) extract inhibits the expression of peroxisome proliferator-activated receptor-γ (PPARγ), CCAAT/enhancer-binding protein-α (CEBPα), perilipin-1, adiponectin, fatty acid binding protein-4 (FABP4), etc., thereby inhibiting the accumulation of lipids in adipocytes. In conclusion, the Geumhwagyu (Hibiscus manihot L.) shows anti-obesity activity by lowering the content of triacylglycerol (TG).

### BRIEF DESCRIPTION OF THE DRAWINGS

FIG. 1 includes Oil Red O-stained cell images showing lipid formation when treated with flower extract (HMF), leaf extract (HMF), stem extract (HMF), and root extract (HMF) of Geumhwagyu (Hibiscus manihot L.) and includes a graph showing the optical density of each cell lysate;

FIG. 2 includes Oil Red O-stained cell images and Western blot test images of Geumhwagyu (Hibiscus manihot L.) leaf extract (HMF), the images showing the optical density, TG content, and control of expression of lipid formation-related proteins (PPARγ, CEBPα, perilipin-1, adiponectin, FABP4) etc.) of cell lysates;

FIG. 3 shows the results of analysis of the effect of Geumhwagyu (Hibiscus manihot L.) leaf extract (HMF) on the lipid formation of adipocytes according to the treatment time, and includes Oil Red O-stained cell images and Western blot test results showing lipid accumulation in cells, the optical density of the cell lysates, and the expression of lipid formation-related proteins;

FIG. 4 shows the lipid accumulation inhibitory effect of Geumhwagyu (Hibiscus manihot L.) leaf extract (HMF) extracted with water and an ethanol aqueous solution, the effect being observed through the Oil Red O staining; and

FIG. 5 shows the lipid accumulation inhibitory effect of Geumhwagyu (Hibiscus manihot L.) leaf extract (HMF) extracted with water and a methanol aqueous solution, the effect being observed through Oil Red O staining.

### DESCRIPTION OF THE PREFERRED EMBODIMENTS

Hereinafter, preferred embodiments of the present disclosure will be described in detail. However, the present disclosure is not limited to the embodiments described herein and may be embodied in other forms. Rather, the embodiments are provided so that this disclosure will be thorough and complete and will fully convey the spirit of the invention to those skilled in the art.

### Example 1: Preparation of Geumhwagyu (Hibiscus Manihot L.) Extract

Flower, leaf, stem, and root extracts of Geumhwagyu (Hibiscus manihot L.) were prepared through a method described below. First, the flowers, leaves, stems, and roots of Geumhwagyu (Hibiscus manihot L.) plants grown by E-Farm Co., Ltd., which is an agricultural company located in Punggi, Gyeongsangbuk-do, Korea, were washed with distilled water and dried. For each of the dried flowers, leaves, stems, and roots, 10 g of the product pulverized plant part was taken and immersed in water at 40° C. for 12 hours to obtain extracts of the flowers, leaves, stems, and roots of Geumhwagyu (Hibiscus manihot L.). In this case, the volume of the water was about 20 times the volume of the dried pulverized plant part. Thereafter, each of the extracts was filtered and then lyophilized to obtain the final extracts of Geumhwagyu (Hibiscus manihot L.) flowers (HMF), leaves (HMF), stems (HMF), and roots (HMF).

### Example 2: Cytotoxicity Assay

In order to check whether the Geumhwagyu (Hibiscus manihot L.) extracts of the present disclosure obtained in Example 1 were toxic to cells, cytotoxicity was analyzed in vitro.

To this end, 3T3-L1, which is a preadipocyte, was purchased from KCLB and used. After the cells were dispensed in a cell culture flask, 10% fetal bovine serum (FBS) and 1% penicillin-streptomycin (10,000 U/ml) were added to a DMEM medium, and the cells were cultured in an incubator maintained at 37° C., 5% CO₂, and 95% humidity conditions, and then used. For cytotoxicity assay, the MTT assay was used. The method uses a principle that dehydrogenases in mitochondria of cells that are normal in their metabolism reduce a yellow water-soluble of tetrazolium salt [3-(4,5-dimethylthiazol-2-yl)-2-5-diphenyltrazolium bromide] (MTT) to non-soluble dark purple MTT formazan crystals. The optical density of the crystals was measured at an appropriate wavelength, for example, mainly 500 to 600 nm, for the evaluation of cytotoxicity.

First, the preadipocytes (3T3-L1) were dispensed into 96 wells at a concentration of 1×10⁵ cells/well and then cultured for 24 hours. Next, the cultured cells were treated with each of the Geumhwagyu (Hibiscus manihot L.) flower, leave, and stem extracts prepared in Example 1, at various concentrations including 0, 50, 100, and 200 μg/mL, and the treated cells were cultured for 24 hours. Next, the MTT was added at a concentration of 1 mg/mL and, and the reaction was performed in an incubator at 37° C. for 2 hours. After the reaction, dimethyl sulfoxide (DMSO) was added, and the optical density was measured at 570 nm using a microplate reader.

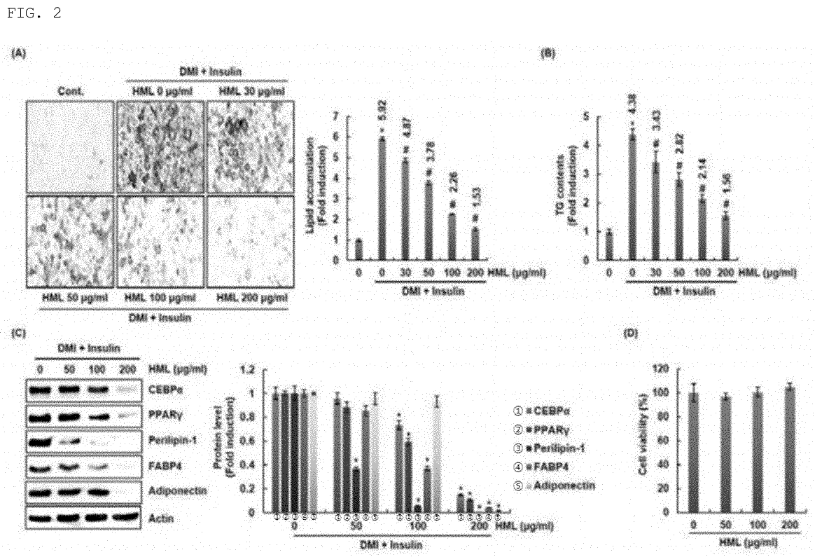

As a result of the analysis, it was confirmed that all of the Geumhwagyu (Hibiscus manihot L.) extracts had no cytotoxicity, and then an experiment described below was performed. The results of only the leave extract are shown in FIG. 2D, but the results of the other extracts are not shown.

### Example 3: Checking Activity of Lipid Accumulation Inhibitory Activity, Triglyceride (TG) Formation Inhibitory Activity, and Inhibitory Activity for Expression of Lipid-Accumulation-Related Proteins

3T3-L1 cells, which are preadipocytes, were dispensed into 96 wells at a concentration of 1×10⁵ cells/well and cultured for 3 days. Next, the cultured cells were differentiated into an MDI medium (adipogenic medium). As the MDI medium, 10% FBS/DMEM treated with 0.5 mM IBMX, 1 μM dexamethasone, and 1 μg/ml insulin was used. This time point was regarded as Day 0 of differentiation, and after 2 days of differentiation, the used medium was replaced with 1 μg/ml of insulin medium. After 2 days of culture, the used medium was replaced with 10% FBS medium, and this condition was maintained until Day 8. The Geumhwagyu (Hibiscus manihot L.) extract was added on Day 0 of differentiation so that the cells were treated with the Geumhwagyu (Hibiscus manihot L.) for 8 days. After 8 days of differentiation, the degree of differentiation of adipocytes and the degree of inhibition of differentiation by the Geumhwagyu (Hibiscus manihot L.) extract were analyzed through Oil Red O staining. After the completion of the differentiation of adipocytes and the treatment with the Geumhwagyu (Hibiscus manihot L.) extract, the cells were washed with 1× phosphate-buffered saline (PBS), followed by addition of 10% (w/v) formalin was, and were fixed at room temperature for 1 hour. After removing the formalin and washing each well used to culture the cells with 60% (v/v) isopropanol, the isopropanol was completely removed under a hood. An Oil Red O solution was added to the dried cell culture wells, and the wells left intact for 10 minutes and then washed with distilled water. Next, adipocyte differentiation was observed through microscopic imaging. The substances remaining in the stained cell culture wells were dissolved in 100% isopropanol, and the optical density of the obtained solution was measured at 500 nm. The amount of triglyceride (TG) in the adipocytes was measured using Triglyceride Assay Kit Quantification, ab65336, which is a commercially available TG assay kit.

For the analysis of proteins, 3T3-L1 cells, which are preadipocytes, were dispensed into 24 wells at a concentration of 1×10⁵ cells/well, cultured for 3 days, and differentiated with a MDI medium (adipogenic medium). This time point was regarded as Day 0 of differentiation, and after 2 days of differentiation, the used medium was replaced with 1 μg/ml of an insulin medium. After 2 days of culture, the used medium was replaced with a 10% FBS medium, and this condition was maintained until Day 8. The Geumhwagyu (Hibiscus manihot L.) extract was added on Day 0 of differentiation so that the cells were treated with the Geumhwagyu (Hibiscus manihot L.) for 8 days. After 8 days of differentiation, the cells were harvested and the expression of lipid accumulation-related proteins was investigated through Western blot analysis.

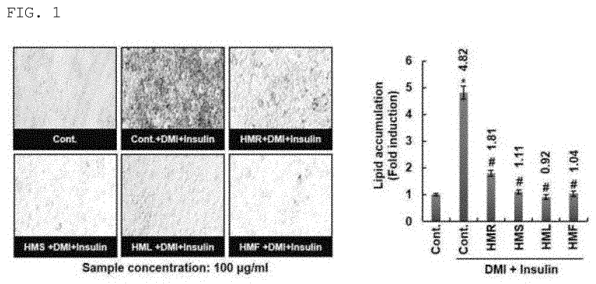

The test results are summarized in FIGS. 1 to 3.

FIG. 1 includes Oil Red O-stained cell images showing lipid formation when treated with flower extract (HMF), leaf extract (HMF), stem extract (HMF), and root extract (HMF) of Geumhwagyu (Hibiscus manihot L.) and includes a graph showing the optical density of each cell lysate. Referring to FIG. 1, it is confirmed that the extracts of Geumhwagyu (Hibiscus manihot L.) roots, stems, leaves, and flowers inhibit lipid formation.

FIG. 2 includes Oil Red O-stained cell image for showing lipid accumulation when treated with an extract of Geumhwagyu (Hibiscus manihot L.) leaves, graphs showing the optical density and TG content of each cell lysate, and Western blot test images showing control of expression of lipid formation-related proteins. Referring to FIG. 2, it is confirmed that the Geumhwagyu (Hibiscus manihot L.) leaf extract inhibits the formation of lipid, the accumulation of TG, and the expression of PPARγ, CEBPα, Perilipin-1, Adiponectin, FABP4, etc., which are proteins associated with lipid formation, in a concentration-dependent manner.

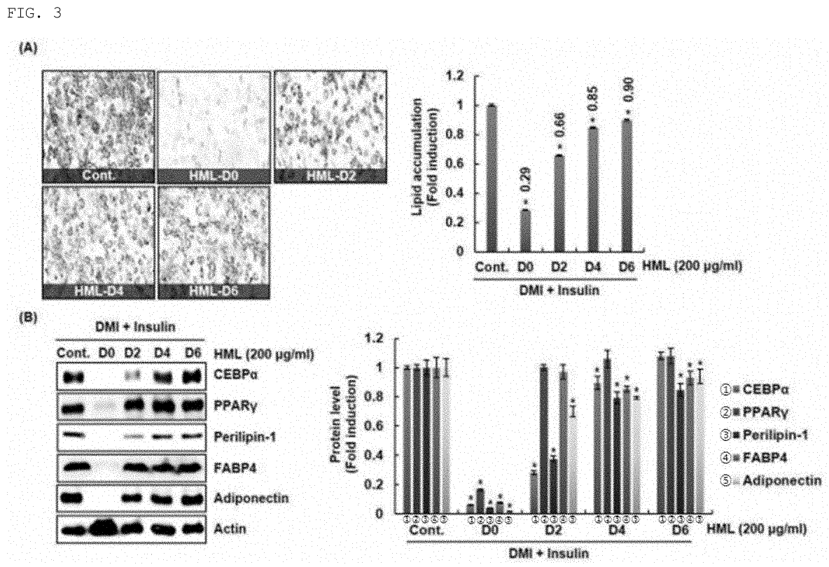

FIG. 3 shows the results of analysis of the effect of Geumhwagyu (Hibiscus manihot L.) leaf extract on the lipid formation of adipocytes according to the treatment time. FIG. 3 includes Oil Red O-stained cell images and Western blot test results showing lipid accumulation, the optical density of each cell lysate, and the expression of lipid formation-related proteins. The Geumhwagyu (Hibiscus manihot L.) leaf extract inhibited lipid accumulation, TG content, and expression of PPARγ, CEBPα, Perilipin-1, Adiponectin, and FABP4, which are proteins associated with lipid accumulation, in a treatment time-dependent manner.

From these results, it was confirmed that when cells were treated with Geumhwagyu (Hibiscus manihot L.) extract after differentiation of the cells from preadipocytes to adipocytes, the lipid accumulation inhibitory effect was about 60% or more, preferably about 60% to 80% at a concentration of 50 μg/ml or more with respect to the adipogenic cells. In addition, the suppression of TG content and the suppression of the expression of lipid formation-related proteins prove that the Geumhwagyu (Hibiscus manihot L.) extract has anti-obesity activity even in the late stage of lipid accumulation in adipocytes.

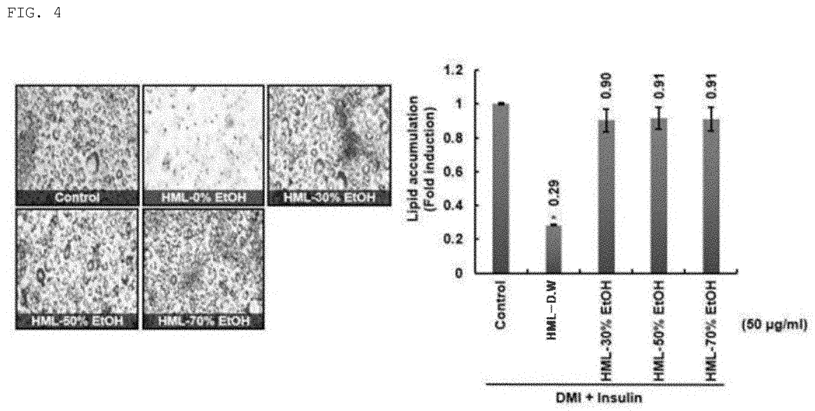

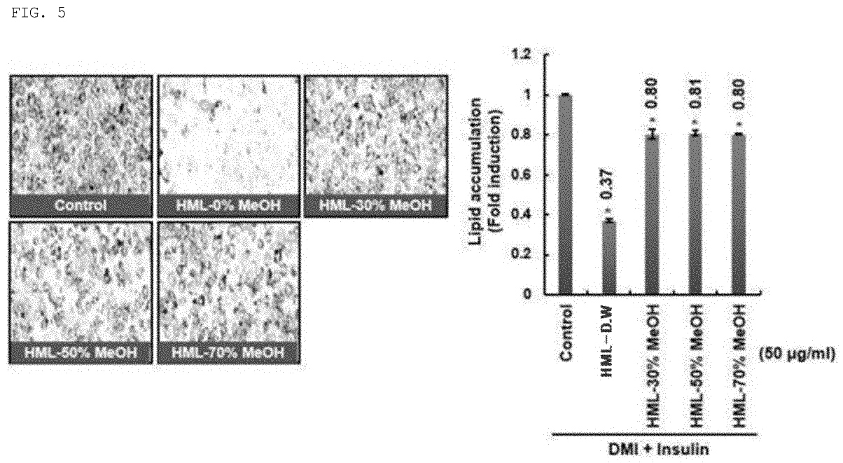

On the other hand, the effect of the Geumhwagyu (Hibiscus manihot L.) leaf extract was compared among the ethanol aqueous solution extract, the methanol aqueous solution extract, and the water extract, and the comparison results are shown in FIGS. 4 and 5. The ethanol aqueous solution extract and the methanol aqueous solution extract were prepared through extraction using an extraction solvent instead of water, followed by distillation under reduced pressure.

Referring to FIGS. 4 and 5, the lipid accumulation inhibitory effect was about 60% or more and preferably about 60% to 70% (v/v) when 50 μg/ml of the water extract was used. That is, the water extract exhibited the remarkable anti-obesity effect. On the other hand, the ethanol 30% to 70% (v/v) aqueous solution extract and the methanol 30% to 70% (v/v) aqueous solution extract exhibited only about 10% of the lipid accumulation inhibitory effect at the same concentration, so the effect was insignificant.

### <Example 4: Checking Weight Loss Efficacy

Three 1-year-old male Pomeranians (having an average weight of about 4 kg) which were neutered at 6 months of age, were fed Royal Canin mini (in-door) feed in an amount according to their body weight for 1 month. In this case, 100 mg of Geumhwagyu (Hibiscus manihot L.) water extract powder was sprinkled on the feed once a day. As a result, the appropriate weight loss effect was confirmed without negatively affecting the health of the test animals during the test period.

What is claimed is:

1. A method of treating obesity for companion animals, comprising:
administering an anti-obesity functional feed composition containing Geumhwagyu (Hibiscus manihot L.) extract or a fraction thereof as an active ingredient to a subject.

2. The method of claim 1, wherein the extract is extract of Geumhwagyu (Hibiscus manihot L.) flowers, stems, leaves, or roots.

3. The method of claim 1, wherein a solvent used to obtain the extract is water, hot water, C₁₋₄ alcohol, or a mixed solution thereof.

4. The method of claim 1, wherein the extract has an effect of inhibiting expression of Peroxisome proliferator-activated receptor-γ (PPARγ), CCAAT/enhancer-binding protein-α (CEBPα), Perilipin-1, Adiponectin, or FABP4 (Fatty acid binding protein-4 which is a protein associated with lipid accumulation in adipocytes.

5. A method of treating obesity for companion animals, comprising:
administering an additive for an anti-obesity functional feed composition containing Geumhwagyu (Hibiscus manihot L.) extract or a fraction thereof as an active ingredient to a subject.

6. The method of claim 5, wherein the extract is extract of Geumhwagyu (Hibiscus manihot L.) flowers, stems, leaves, or roots.

7. The method of claim 5, wherein a solvent used to obtain the extract is water, hot water, C₁₋₄ alcohol, or a mixed solution thereof.

8. The method of claim 5, wherein the extract has an effect of inhibiting expression of Peroxisome proliferator-activated receptor-γ (PPARγ), CCAAT/enhancer-binding protein-α (CEBPα), Perilipin-1, Adiponectin, or FABP4 (Fatty acid binding protein-4 which is a protein associated with lipid accumulation in adipocytes.
